(19) United States
(12) Patent Application Publication
(10) Pub. No.: US 2013/0018261 A1
(43) Pub. Date: Jan. 17, 2013

(54) Ultrasonic Apparatus Comprising Therapeutic Ultrasonic Transducer Detachably Coupled to Diagnostic Ultrasonic Transducer

(71) Applicants: Jong Seob Jeong, Seoul (KR); SungMin Kim, Goyang-si (KR); Jun Young Park, Seoul (KR)

(72) Inventors: Jong Seob Jeong, Seoul (KR); SungMin Kim, Goyang-si (KR); Jun Young Park, Seoul (KR)

(73) Assignee: Dongguk University, Industry-Academic Cooperation Foundation, Seoul (KR)

(21) Appl. No.: 13/535,687

(22) Filed: Jun. 28, 2012

(30) Foreign Application Priority Data
Jul. 12, 2011 (KR) 10-2011-0068927

(51) Int. Cl. A61N 7/00; A61B 8/13

(52) U.S. Cl. 600/439

(57) ABSTRACT

An ultrasonic apparatus including a therapeutic ultrasonic transducer and a diagnostic ultrasonic transducer which is detachably coupled to the therapeutic ultrasonic transducer is provided. Here, the therapeutic and diagnostic ultrasonic transducers can be mounted to be independently replaceable with new ones. The therapeutic ultrasonic transducer and the diagnostic ultrasonic transducer, both of which constitute the ultrasonic apparatus, can be independently exchanged, transducers having various frequency bands and focusing distances can be combined, and damage to the diagnostic ultrasonic transducer can be prevented by preventing heat generated from therapeutic ultrasonic waves from being transferred to the diagnostic ultrasonic transducer.

### CROSS-REFERENCE TO RELATED APPLICATION

This application claims priority to and the benefit of Korean Patent Application No. 2011-0068927, filed Jul. 12, 2011, the disclosure of which is incorporated herein by reference in its entirety.

### BACKGROUND

1. Field of the Invention

The present invention relates to an ultrasonic apparatus, and more particularly, to an ultrasonic apparatus including a therapeutic ultrasonic transducer and a diagnostic ultrasonic transducer, which are detachably coupled.

2. Discussion of Related Art

In general, an ultrasonic apparatus used for ultrasonic image-guided high intensity focused ultrasound (HIFU) surgeries includes a diagnostic ultrasonic transducer configured to irradiate a patient with ultrasonic waves and provide an internal shape of a part to be diagnosed as a two- or three-dimensional image and a therapeutic ultrasonic transducer configured to irradiate a diseased part of a patient with focused high intensity ultrasonic waves to destroy or necrotize diseased tissues.

By way of example, Japanese Patent Publication No. 2003-199761 discloses an ultrasonic therapeutic apparatus including a probe configured to transmit and receive ultrasonic waves to/from the inside of a subject and used to collect and diagnose ultrasonic tomographic images of a part to be treated, a therapeutic ultrasonic transducer configured to irradiate the part to be treated with high-energy ultrasonic waves, an ultrasonic picture display device configured to display an ultrasonic tomographic picture obtained using the probe, and a means configured to indicate a position of the part to be treated on a display screen of the ultrasonic picture display device. Here, the ultrasonic picture display device is characterized in that it uses a touch panel-type ultrasonic picture display device and marks and sets the position of the part to be treated on the display screen of the touch panel of the ultrasonic picture display device using a stylus.

However, the diagnostic ultrasonic transducer and the therapeutic ultrasonic transducer, which constitute the ultrasonic apparatus for ultrasonic image-guided HIFU surgeries, are formed integrally with each other, and thus function to obtain ultrasonic images or treat a diseased part by allowing a corresponding ultrasonic transducer to work under desired conditions, for example, therapeutic or diagnostic conditions.

In particular, the ultrasonic image-guided HIFU surgeries are performed by repeatedly performing procedures of first diagnosing an image of a part to be treated and then performing ultrasound therapy or performed at the same time as the real-time diagnosis and treatment. As a result, the ultrasonic apparatus including both the therapeutic ultrasonic transducer and the diagnostic ultrasonic transducer has been generally used in the art.

However, since the therapeutic ultrasonic transducer of the ultrasonic apparatus in which the therapeutic ultrasonic transducer is formed integrally with the diagnostic ultrasonic transducer generates high-power ultrasonic waves, the therapeutic ultrasonic transducer has a shorter lifespan than the diagnostic ultrasonic transducer, and although the diagnostic ultrasonic transducer has a relatively longer lifespan, it is replaced along with the therapeutic ultrasonic transducer whose lifespan has completed.

Particularly, when the ultrasonic apparatus in which the therapeutic ultrasonic transducer is formed integrally with the diagnostic ultrasonic transducer is used in the field of plastic surgery, the therapeutic ultrasonic transducer is changed with a new one after the transducer is repeatedly used a plurality of times, for example, 10 times. In this case, the use of an expensive ultrasonic transducer results in an increase in charges for surgical operations.

In addition, when the therapeutic ultrasonic transducer and the diagnostic ultrasonic transducer are formed integrally with each other, the diagnostic ultrasonic transducer may be damaged by reflection of high-temperature and high-intensity focused ultrasound energy generated through the use of the therapeutic ultrasonic transducer, thereby reducing the lifespan of the diagnostic ultrasonic transducer.

Also, since the frequencies and focusing distances of the two ultrasonic transducers which constitute an integrated transducer are fixed, it is difficult to vary the frequencies and focusing distances according to the conditions of a subject to be treated, and an integrated transducer having different frequencies and focusing distances is purchased, when necessary.

Furthermore, since the ultrasonic apparatus in which the therapeutic ultrasonic transducer is formed integrally with the diagnostic ultrasonic transducer uses only one ground connection, the image quality may be degraded due to the strong ground noises generated in the therapeutic ultrasonic transducer when the diagnostic ultrasonic transducer is used to take an image of a patient.

### SUMMARY OF THE INVENTION

The present invention is directed to providing an ultrasonic apparatus including a therapeutic ultrasonic transducer configured to provide high-intensity focused ultrasonic waves used for therapeutic purposes and a diagnostic ultrasonic transducer detachably coupled to the therapeutic ultrasonic transducer and configured to provide ultrasonic waves so as to obtain ultrasonic images. Here, the therapeutic ultrasonic transducer and the diagnostic ultrasonic transducer can be mounted to be independently replaceable with new ones.

One aspect of the present invention provides an ultrasonic apparatus including a therapeutic ultrasonic transducer and a diagnostic ultrasonic transducer which is detachably coupled to the therapeutic ultrasonic transducer. Here, the therapeutic and diagnostic ultrasonic transducers are mounted to be independently replaceable with new ones.

Also, the ultrasonic apparatus may further include a heat insulating unit provided between the therapeutic ultrasonic transducer and the diagnostic ultrasonic transducer to prevent heat generated with the operation of the therapeutic ultrasonic transducer from being transferred into the diagnostic ultrasonic transducer.

In the ultrasonic apparatus, the therapeutic ultrasonic transducer and the diagnostic ultrasonic transducer may also be independently grounded.

Also, since the therapeutic and diagnostic transducers having different frequencies and/or focusing distances are separated to be interchangeable with each other, various combinations of the therapeutic and diagnostic transducers may be provided in the ultrasonic apparatus.

Also, the ultrasonic apparatus may further include a lid which is coupled to one side of the diagnostic ultrasonic transducer to prevent ultrasonic waves generated from the therapeutic ultrasonic transducer from being reflected and transmitted to the diagnostic ultrasonic transducer.

### BRIEF DESCRIPTION OF THE DRAWINGS

The above and other objects, features and advantages of the present invention will become more apparent to those of ordinary skill in the art by describing in detail the exemplary embodiments thereof with reference to the attached drawings in which:

FIG. 1 is a configuration diagram showing one exemplary embodiment of an ultrasonic apparatus according to the present invention,

FIG. 2 is a cross-sectional view showing a section of the ultrasonic apparatus according to the present invention,

FIG. 3 is a configuration diagram showing another exemplary embodiment of the ultrasonic apparatus according to the present invention, and

FIG. 4 is a configuration diagram showing a section of the ultrasonic apparatus according to another exemplary embodiment of the present invention.

### DETAILED DESCRIPTION OF EXEMPLARY EMBODIMENTS

Hereinafter, the exemplary embodiments of the present invention will be described in detail. However, the present invention is not limited to the embodiments disclosed below, but can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and similarly, a second element could be termed a first element, without departing from the scope of exemplary embodiments. The terms “and/or” include any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of the exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, the exemplary embodiments of the present invention will be described in detail below. To aid in understanding the present invention, like numbers refer to like elements throughout the description of the figures, and the description of the same elements will not be reiterated.

The present invention provides an ultrasonic apparatus including a therapeutic ultrasonic transducer and a diagnostic ultrasonic transducer which is detachably coupled to the therapeutic ultrasonic transducer. Here, the therapeutic and diagnostic ultrasonic transducers are mounted to be independently replaceable with new ones.

The ultrasonic apparatus according to the present invention includes a therapeutic ultrasonic transducer and a diagnostic ultrasonic transducer. In this case, the therapeutic and diagnostic ultrasonic transducers are not particularly limited as long as they can be detachably coupled to each other.

In this case, the ultrasonic apparatus may be used for the ultrasonic image-guided HIFU surgeries.

The therapeutic ultrasonic transducer according to the present invention may be used without limitation as long as it can provide ultrasonic waves used for therapeutic purposes, for example, high intensity-focused ultrasonic waves.

The diagnostic ultrasonic transducer according to the present invention may be used without limitation as long as it can provide ultrasonic waves so as to obtain ultrasonic images.

Here, the shapes of the therapeutic ultrasonic transducer and the diagnostic ultrasonic transducer are shapes of typical transducers known in the art. The shapes of the ultrasonic transducers are not particularly limited, but the use of an array-type or single-element transducer may be recommended.

Hereinafter, the present invention will be described in further detail with reference to the accompanying drawings. However, it should be understood that the description proposed herein is merely a preferable example for the purpose of illustration only, not intended to limit the scope of the invention.

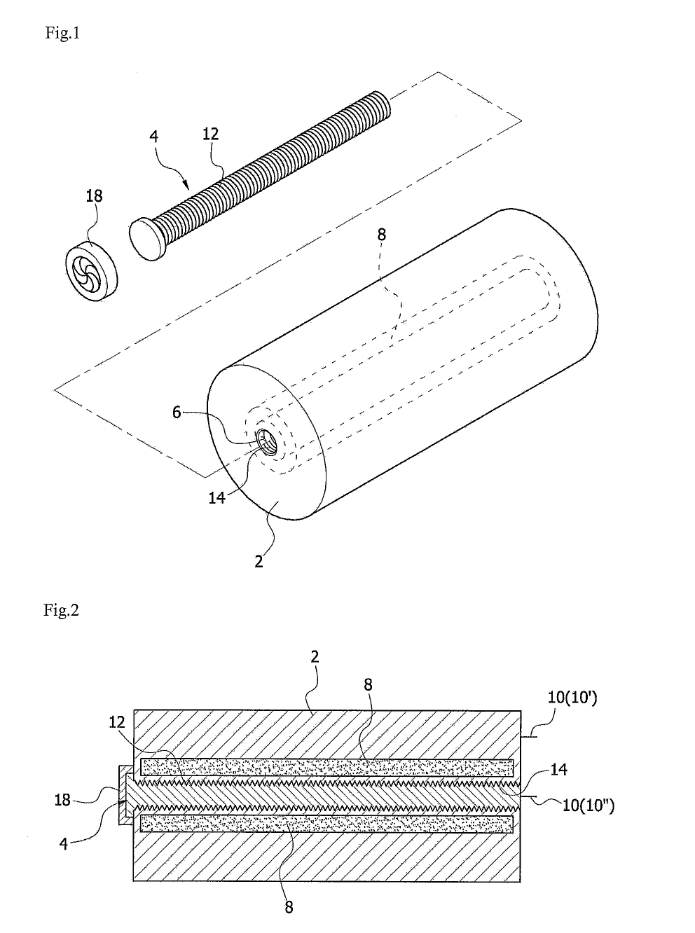

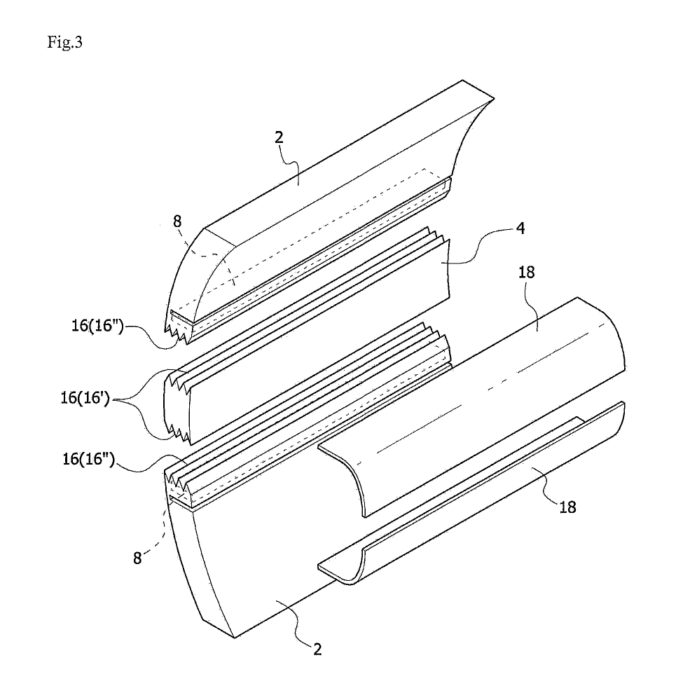

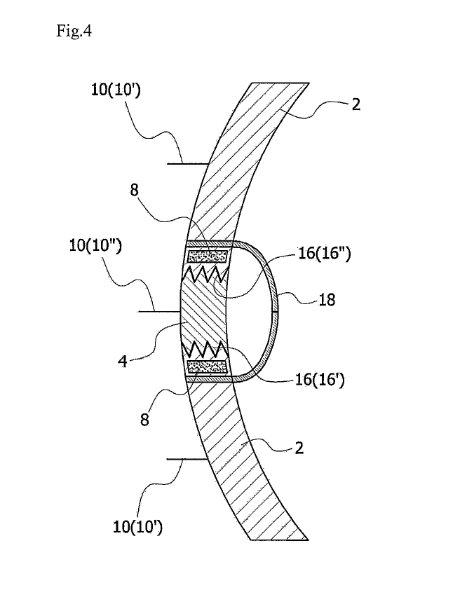

FIG. 1 is a configuration diagram showing one exemplary embodiment of an ultrasonic apparatus according to the present invention, FIG. 2 is a cross-sectional view showing a section of the ultrasonic apparatus according to the present invention, FIG. 3 is a configuration diagram showing another exemplary embodiment of the ultrasonic apparatus according to the present invention, and FIG. 4 is a configuration diagram showing a section of the ultrasonic apparatus according to another exemplary embodiment of the present invention.

As shown in FIGS. 1 to 4, the ultrasonic apparatus according to the present invention includes a therapeutic ultrasonic transducer 2 and a diagnostic ultrasonic transducer 4 that are detachably coupled. Here, the therapeutic ultrasonic transducer 2 and the diagnostic ultrasonic transducer 4 are configured to be replaced, for example, independently replaceable, with new ones.

The therapeutic ultrasonic transducer 2 according to the present invention may be used without limitation as long as it can provide ultrasonic waves used for therapeutic purposes, for example, high intensity-focused ultrasonic waves.

In particular, the therapeutic ultrasonic transducer 2 functions to convert electric energy into ultrasonic energy, for example, HIFU energy. Here, the typical ultrasonic transducers known in the art are used without limitation as long as they can be used for these purposes.

Also, the shapes of the therapeutic ultrasonic transducer 2 are not particularly limited as long as the therapeutic ultrasonic transducer 2 can generate ultrasonic waves, for example, high intensity-focused ultrasonic waves.

The diagnostic ultrasonic transducer 4 according to the present invention functions to provide ultrasonic waves which may be used to obtain ultrasonic images. Here, the typical ultrasonic transducers known in the art are used without limitation as long as they can be used for these purposes.

In particular, the diagnostic ultrasonic transducer 4 functions to convert electric energy into ultrasonic energy, for example, ultrasonic energy for obtaining images. Here, the typical ultrasonic transducers known in the art are used without limitation as long as they can be used for this purpose, and the shapes of the ultrasonic transducers are not particularly limited either.

Here, the connection pattern between the therapeutic ultrasonic transducer 2 and the diagnostic ultrasonic transducer 4 is not particularly limited.

According to one specific exemplary embodiment in which the therapeutic ultrasonic transducer 2 and the diagnostic ultrasonic transducer 4 are coupled to each other, a heat insulating unit 8 configured to prevent heat transfer between the therapeutic ultrasonic transducer 2 and the diagnostic ultrasonic transducer 4 may be provided between the therapeutic ultrasonic transducer 2 and the diagnostic ultrasonic transducer 4 to prevent heat generated with the operation of the therapeutic ultrasonic transducer 2 from being transferred into the diagnostic ultrasonic transducer 4.

Here, the heat insulating unit 8 may be used without particular limitation as long as it can be formed of a heat-insulating material that can prevent heat transfer. Also, the shapes of the heat insulating unit 8 may be selectively varied according to the shapes of the therapeutic ultrasonic transducer 2 and the diagnostic ultrasonic transducer 4.

Also, the heat insulating unit 8 may be configured so that a contact surface between the therapeutic ultrasonic transducer 2 and the diagnostic ultrasonic transducer 4 can be coated with a heat-insulating material.

According to another specific exemplary embodiment, the therapeutic ultrasonic transducer 2 and the diagnostic ultrasonic transducer, which constitute the ultrasonic apparatus according to the present invention, may be configured to be independently grounded.

This may prevent ground noises generated in the therapeutic ultrasonic transducer 2 from interfering with the diagnostic ultrasonic transducer 4 in use, thereby enabling ultrasonic images with better quality to be obtained and an electromagnetic shielding effect of the shielding electromagnetic waves transmitted from the therapeutic ultrasonic transducer 2 to the diagnostic ultrasonic transducer can be achieved.

According to still another specific exemplary embodiment, the ultrasonic apparatus according to the present invention may further include a lid 18 which is coupled to one side of the diagnostic ultrasonic transducer 4 to prevent ultrasonic waves generated from the therapeutic ultrasonic transducer 2 from being reflected and transmitted to the diagnostic ultrasonic transducer 4.

Here, when the HIFU energy generated from the therapeutic ultrasonic transducer 2 is transmitted to the diagnostic ultrasonic transducer 4, the lifespan of the diagnostic ultrasonic transducer may be shortened due to the HIFU energy.

In this case, when the lid 18 is mounted on one side, for example, one front side, of the diagnostic ultrasonic transducer 4 and the diagnostic ultrasonic transducer 4 is not used, the lid 18 is attached to the diagnostic ultrasonic transducer 4 or closed, and is detached from the diagnostic ultrasonic transducer 4 in use or opened, thereby selectively preventing the HIFU energy from being transmitted into the diagnostic ultrasonic transducer 4

In this case, the lid 18 may be used without limitation as long as it can prevent reflected ultrasonic energy from passing through the diagnostic ultrasonic transducer 4.

Also, the lid 18 may be manually handled to be attached or detached to/from the diagnostic ultrasonic transducer 4. However, the lid 18 may be made in the form of a shutter or diaphragm, and thus be configured so that the lid 18 can be automatically opened for the use of the diagnostic ultrasonic transducer 4 and automatically closed when the diagnostic ultrasonic transducer 4 is not used.

Meanwhile, since the therapeutic ultrasonic transducer 2 and the diagnostic ultrasonic transducer 4 which constitute the ultrasonic apparatus according to the present invention are independently replaceable with new ones, the transducers having a variety of frequencies and focusing distances may be selectively adopted and used for HIFU surgery according to information of a diseased part to be treated, for example, information such as a size and position of the diseased part.

The ultrasonic apparatus having such a configuration according to the present invention may be configured in a shape widely used in the art for the ultrasonic apparatus, for example, a ring shape, a shape extending in a longitudinal direction, a shape having a fan-shaped front, an internally curved planar shape, etc. This embodiment will be described by way of example, as follows. However, the present invention is not limited to the following embodiments.

According to one exemplary embodiment of the above-described ultrasonic apparatus, the ultrasonic apparatus according to the present invention may include a therapeutic ultrasonic transducer 2 which has a cylindrical shape extending in a longitudinal direction and has grooves 6 formed therein to insert a diagnostic transducer 4 into one central side of the cylindrical shape; and a diagnostic ultrasonic transducer 4 which extends in a longitudinal direction and is inserted into and coupled to the grooves 6 of the therapeutic ultrasonic transducer 2.

Here, the diagnostic ultrasonic transducer 4 extending in a longitudinal direction may have male threads 12 formed along a circumferential surface thereof in a longitudinal direction, and female threads 14 corresponding to the male threads may be formed in the grooves 6 of the therapeutic ultrasonic transducer 2 into which the diagnostic ultrasonic transducer 4 is inserted.

Also, a heat insulating unit 8 may be provided between the therapeutic ultrasonic transducer 2 and the diagnostic ultrasonic transducer 4 which constitute the ultrasonic apparatus.

In this case, the heat insulating unit 8 may be formed on the periphery of the grooves 6 formed in the therapeutic ultrasonic transducer 2 in the longitudinal direction of the therapeutic ultrasonic transducer 2. Also, the heat insulating unit 8 may be formed on the circumferential surface of the diagnostic ultrasonic transducer 4 in a longitudinal direction. When necessary, the heat insulating unit 8 may be formed on both of the grooves 6 of the therapeutic ultrasonic transducer 2 and the circumferential surface of the diagnostic ultrasonic transducer 4.

The heat insulating unit 8 may be formed in a layered structure made of a heat-insulating material.

Also, a lid 18 may be further provided on one side, for example, one front side, of the diagnostic ultrasonic transducer 4 according to the present invention to prevent ultrasonic waves generated in the therapeutic ultrasonic transducer 2 from being reflected and transmitted into the diagnostic ultrasonic transducer 4.

In this case, the lid 18 is provided in the form of a diaphragm or shutter, and thus may be automatically opened/closed according the use of the diagnostic ultrasonic transducer 4.

According to a specific exemplary embodiment, the therapeutic ultrasonic transducer 2 and the diagnostic ultrasonic transducer 4 which constitute the above-described ultrasonic apparatus may be changed in shape and position according to a user's choice.

Meanwhile, according to another exemplary embodiment of the ultrasonic apparatus of the present invention, the ultrasonic apparatus according to the present invention may include a diagnostic ultrasonic transducer 4 having a hexahedral shape extending in a longitudinal direction, and a therapeutic ultrasonic transducers 2 which are coupled to the upper and lower portions of the diagnostic ultrasonic transducer 4 in a longitudinal direction.

Here, teeth 16 are formed in contact surfaces between the diagnostic ultrasonic transducer 4 and the therapeutic ultrasonic transducers 2 to allow the diagnostic ultrasonic transducer 4 and the therapeutic ultrasonic transducers 2 to engage with each other in a longitudinal direction. As a result, the diagnostic ultrasonic transducer 4 may be configured so that it can be inserted between the two therapeutic ultrasonic transducers 2 along the teeth 16 and coupled to the therapeutic ultrasonic transducers 2.

In this case, when it is arbitrarily assumed that the teeth 10 formed in the diagnostic ultrasonic transducer 4 are first teeth 16′, the teeth 10 formed in the therapeutic ultrasonic transducers 2 may be referred to as second teeth 16″ so as to engage with the first teeth 16′.

Also, heat insulating units 8 may be provided between the therapeutic ultrasonic transducers 2 and the diagnostic ultrasonic transducer 4 that constitute the ultrasonic apparatus.

In this case, the heat insulating unit 8 may be formed in a longitudinal direction of the therapeutic ultrasonic transducer 2 on one side of the therapeutic ultrasonic transducer 2 which comes in contact with the diagnostic ultrasonic transducer 4. Also, the heat insulating unit 8 may be formed in a longitudinal direction on one side of the diagnostic ultrasonic transducer 4 which comes in contact with the therapeutic ultrasonic transducer 2. When necessary, the heat insulating units 8 may be formed on one contact surface between the therapeutic ultrasonic transducers 2 and the diagnostic ultrasonic transducer 4, that is, both contact surfaces of the therapeutic ultrasonic transducers 2 and the diagnostic ultrasonic transducer 4.

The heat insulating units 8 may be in a layered structure formed of a heat-insulating material.

Also, a lid 18 may be further provided on one side, for example, one front side, of the diagnostic ultrasonic transducer 4 to prevent ultrasonic waves generated in the therapeutic ultrasonic transducers 2 from being reflected and transmitted into the diagnostic ultrasonic transducer 4.

In this case, the lid 18 is provided in the form of a shutter, and thus may be automatically opened/closed according the use of the diagnostic ultrasonic transducer 4.

Also, the lid 18 may be inserted and fixed on one side of the therapeutic ultrasonic transducers 2 or the diagnostic ultrasonic transducer 4, for example, one side in which the grooves are formed.

Meanwhile, the grounds 10 are coupled respectively to the sides of the therapeutic ultrasonic transducers 2 and the diagnostic ultrasonic transducer 4 that constitute the above-mentioned ultrasonic apparatus according to the present invention, and thus may be configured so that the use of the therapeutic ultrasonic transducers 2 and the diagnostic ultrasonic transducer 4 cannot prevent ground noises from affecting the grounds 10.

In this case, when it is arbitrarily assumed that the grounds 10 coupled to the therapeutic ultrasonic transducers 2 are first grounds 10′, the ground 10 coupled to the diagnostic ultrasonic transducer 4 may be referred to as a second ground 10″.

According to a specific exemplary embodiment, the therapeutic ultrasonic transducers 2 and the diagnostic ultrasonic transducer 4 which constitute the above-described ultrasonic apparatus may be changed in the shape and position according to a user's choice.

Also, the entire shape of the above-described ultrasonic apparatus may have an internally curved planar shape extending in a longitudinal direction.

For the ultrasonic apparatus according to the present invention, the therapeutic ultrasonic transducer and the diagnostic ultrasonic transducer that constitute the ultrasonic apparatus may be independently replaceable with new ones, and damage to the diagnostic ultrasonic transducer may be prevented by preventing heat generated from therapeutic ultrasonic waves from being transferred to the diagnostic ultrasonic transducer.

According to the present invention, the grounds may also be independently connected to the therapeutic and diagnostic ultrasonic transducers so that the ground noises generated in the therapeutic ultrasonic transducer can be prevented from interfering with the ultrasonic waves in use by the diagnostic ultrasonic transducer.

In addition, according to the present invention, since the therapeutic and diagnostic transducers having different frequencies and/or focusing distances are separated to be interchangeable with each other, various combinations of the therapeutic and diagnostic transducers may be provided, which leads to effective medical treatments and a decrease in costs.

Furthermore, according to the present invention, a lid through which ultrasonic energy does not pass is mounted on one side of the diagnostic ultrasonic transducer so that the reflected HIFU energy can be prevented from being transferred to the diagnostic ultrasonic transducer in use of the therapeutic ultrasonic transducer, thereby preventing damage to the diagnostic ultrasonic transducer.

While the invention has been shown and described with reference to certain exemplary embodiments thereof, it will be understood by those skilled in the art that various changes in form and details may be made therein without departing from the scope of the invention as defined by the appended claims.

What is claimed is:

1. An ultrasonic apparatus comprising:
a therapeutic ultrasonic transducer; and
a diagnostic ultrasonic transducer which is detachably coupled to the therapeutic ultrasonic transducer,
wherein the therapeutic and diagnostic ultrasonic transducers are mounted to be independently replaceable with new ones.

2. The ultrasonic apparatus of claim 1, further comprising:
a heat insulating unit provided between the therapeutic ultrasonic transducer and the diagnostic ultrasonic transducer to prevent heat generated with the operation of the therapeutic ultrasonic transducer from being transferred into the diagnostic ultrasonic transducer.

3. The ultrasonic apparatus of claim 1, wherein the therapeutic ultrasonic transducer and the diagnostic ultrasonic transducer are independently grounded.

4. The ultrasonic apparatus of claim 1, wherein the therapeutic ultrasonic transducer or the diagnostic ultrasonic transducer is an array-type or single element transducer.

5. The ultrasonic apparatus of claim 1, further comprising:
a lid which is coupled to one side of the diagnostic ultrasonic transducer to prevent ultrasonic waves generated from the therapeutic ultrasonic transducer from being reflected and transmitted to the diagnostic ultrasonic transducer.

6. The ultrasonic apparatus of claim 5, wherein the lid is in the form of a diaphragm or shutter.

7. The ultrasonic apparatus of claim 5, wherein the lid is automatically opened and closed.

8. The ultrasonic apparatus of claim 1, wherein the ultrasonic apparatus comprises:
a therapeutic ultrasonic transducer that has a cylindrical shape extending in a longitudinal direction and has grooves formed therein to be able to insert a diagnostic transducer into one central side of the cylindrical shape; and
the diagnostic ultrasonic transducer which extends in a longitudinal direction and is inserted into and coupled to the grooves of the therapeutic ultrasonic transducer.

9. The ultrasonic apparatus of claim 8, wherein male threads are formed along a circumferential surface of the diagnostic ultrasonic transducer, and female threads corresponding to the male threads are formed in the grooves of the therapeutic ultrasonic transducer into which the diagnostic ultrasonic transducer is inserted.

10. The ultrasonic apparatus of claim 1, wherein the ultrasonic apparatus comprises:
a diagnostic ultrasonic transducer that has a cylindrical shape extending in a longitudinal direction and has grooves formed therein to be able to insert a therapeutic transducer into one central side of the cylindrical shape; and
the therapeutic ultrasonic transducer which extends in a longitudinal direction and is inserted into and coupled to the grooves of the diagnostic ultrasonic transducer.

11. The ultrasonic apparatus of claim 10, wherein male threads are formed along a circumferential surface of the therapeutic ultrasonic transducer, and female threads corresponding to the male threads are formed in the grooves of the diagnostic ultrasonic transducer into which the therapeutic ultrasonic transducer is inserted.

12. The ultrasonic apparatus of claim 1, wherein the ultrasonic apparatus comprises:
a diagnostic ultrasonic transducer which has a hexahedral shape extending in a longitudinal direction; and
a therapeutic ultrasonic transducer which extends in longitudinal direction and is coupled to the upper and lower portions of the diagnostic ultrasonic transducer in a longitudinal direction.

13. The ultrasonic apparatus of claim 12, wherein teeth patterns are formed in a contact surface between the diagnostic ultrasonic transducer and the therapeutic ultrasonic transducer in a longitudinal direction.

14. The ultrasonic apparatus of claim 1, wherein the ultrasonic apparatus comprises:
a therapeutic ultrasonic transducer which has a hexahedral shape extending in a longitudinal direction; and
a diagnostic ultrasonic transducer which extends in longitudinal direction and is coupled to the upper and lower portions of the therapeutic ultrasonic transducer in a longitudinal direction.
